(19) United States
(12) Patent Application Publication
(10) Pub. No.: US 2004/0106910 A1
(43) Pub. Date: Jun. 3, 2004

(54) Dual layer absorbent structure having an intake/transfer carrier sheet and method of making

(76) Inventors: Susan M. Vercauteren, Greenville, WI (US); Cindy L. Price, Appleton, WI (US); Joseph DiPalma, Neenah, WI (US); Chris Boudrie, Appleton, WI (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/307,895

(22) Filed: Dec. 2, 2002

(51) Int. Cl. A61F 13/15; A61F 13/20

(52) U.S. Cl. 604/378

(57) ABSTRACT

An absorbent article includes a body side liner and a garment side baffle having an intake/transfer carrier sheet therein. The intake/transfer layer forms a carrier sheet for one or more absorbent layers within the envelope formed by the body side liner and garment side baffle. The carrier sheet supports a first absorbent layer that contacts a second absorbent layer.

### TECHNICAL FIELD

A pad having dual layer structure for receiving a liquid. More particularly, a pad having dual layer structure for receiving a liquid with an intake/transfer layer as a carrier layer for a fluff based absorbent containing particulates.

### TECHNICAL BACKGROUND

Disposable absorbent articles are used in several products such as incontinent products, menstrual pads, and diapers. The products are formed of one or more layers often containing super absorbent material. However, when super absorbent material is used, it is be wrapped or overlaid with an envelope of material such as tissue. Tissue can interfere with proper absorption of insults, and contribute negatively to flowback issues. Furthermore, after processing or debulking of the article during manufacturing, the envelope of tissue can interfere with bonding between the multiple layers throughout the article and lessen the integration of the construction.

Accordingly, what is needed is an improved absorbent article and an improved method of making an absorbent article.

### SUMMARY

An absorbent article is provided that includes a moisture permeable body side liner that has an outer body side liner surface and an inner body side liner surface. The absorbent article further includes an intake/transfer layer with a first intake layer surface and a second intake layer surface. The intake/transfer layer is disposed in fluid communication with the moisture permeable body side liner. The absorbent article further includes a body side absorbent layer disposed in fluid communication with the intake/transfer layer, where the body side absorbent layer contacts the intake/transfer layer. A garment side absorbent layer is also included, and the garment side absorbent layer is in fluid communication with the body side absorbent layer. A substantially moisture impermeable garment side baffle provides a second outer surface of the absorbent article. The intake/transfer layer has a footprint that is at least as large as the body side absorbent layer footprint.

Several options for the absorbent article are as follows. For instance, in one option, the body side absorbent layer and the garment side absorbent layer includes about 70% fluff material and about 30% super absorbent material by weight. In another option, the body side absorbent layer includes about 60-90% fluff material and about 40-10% super absorbent material by weight, or any particular percentages between the ranges specified. In another option, the intake/transfer layer footprint is larger than the body side absorbent layer footprint. Optionally, the intake/transfer layer is formed of lofty, non-woven material, or a bi-component fiber such as polyolefin and ethylene alkyl acrylate copolymer.

In another embodiment, an absorbent article includes a moisture permeable body side liner that has an outer body side liner surface and an inner body side liner surface. The article further includes an intake/transfer layer that is disposed in fluid communication with the moisture permeable body side liner. Dual absorbent layers of particulate material are also included in the article, where the dual layers include a first absorbent layer and a second absorbent layer. A first surface of the first absorbent layer is disposed in contact with the intake/transfer layer, and the first absorbent layer is carried by the intake/transfer layer. The article further includes a substantially moisture impermeable garment side baffle that has an outer garment side surface and an inner garment side surface. An inner garment side surface of the garment side baffle is in contact with the second surface of the second absorbent layer. The intake/transfer layer has a footprint that is at least as large as a first absorbent layer footprint.

Several options for the article are as follows. For instance, in one option, the intake/transfer layer has a first footprint, and the first absorbent layer has a second footprint, and the first footprint is larger than the second footprint. In yet another option, a layer of adhesive is disposed between the intake/transfer layer and the first surface of the first absorbent layer. Optionally, the intake/transfer layer is not formed of tissue.

In yet another embodiment, a method for forming an absorbent article is provided. The method includes forming a particulate material on a processing line at a forming location, providing a layer of intake/transfer material proximate to the forming location, conveying the layer of intake/transfer material along a conveyor, for example a vacuum conveyor, and depositing a first layer of absorbent material on the layer of intake/transfer material on the conveyor.

Several options for the method are as follows. For instance, in one option, the method further includes forming a layer of adhesive on the layer of intake/transfer material prior to forming the first layer of absorbent material on the layer of intake/transfer material. In yet another option, the method further includes forming a second layer of absorbent material on the first layer of absorbent material, such as by forming the second layer of absorbent material directly on the first layer of absorbent material. The intake/transfer material is formed, for example, in segments, or in a continuous stream of material. Optionally, the first layer of absorbent material has a width the same as or less than the layer of intake/transfer material.

These and other embodiments, aspects, advantages, and features of the present invention will be set forth in part in the description which follows, and in part will become apparent to those skilled in the art by reference to the following description of the invention and referenced drawings or by practice of the invention. The aspects, advantages, and features of the invention are realized and attained by means of the instrumentalities, procedures, and combinations particularly pointed out in the appended claims and their equivalents.

### BRIEF DESCRIPTION OF THE DRAWINGS

FIG. 1 is a top plan view of an absorbent article constructed in accordance with one embodiment;

FIG. 2 is a cross-section taken along A-A of FIG. 1 constructed in accordance with one embodiment; and

FIG. 3 is a block diagram illustrating an apparatus for forming at least a portion of the absorbent article.

### DETAILED DESCRIPTION

The following description includes terms, such as first, second, etc. that are used for descriptive purposes only and are not to be construed as limiting. In the following detailed description, reference is made to the accompanying drawings, which form a part hereof. These drawings show, by way of illustration, specific embodiments in which the invention may be practiced. In the drawings, some of the like numerals describe substantially similar components throughout the several views. These embodiments are described in sufficient detail to enable those skilled in the art to practice the invention. Other embodiments may be used and structural changes may be made without departing from the scope of the several embodiments.

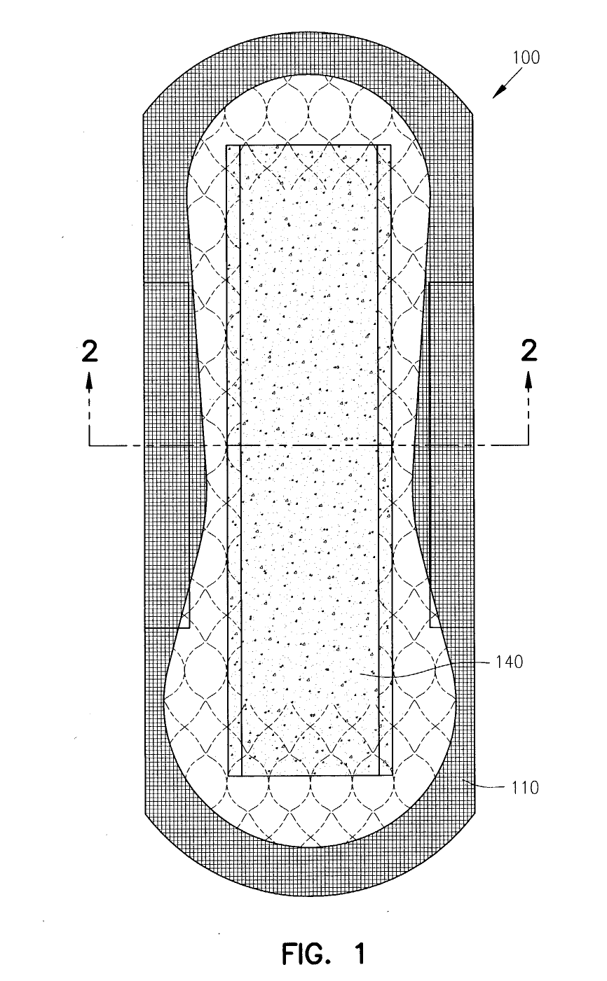

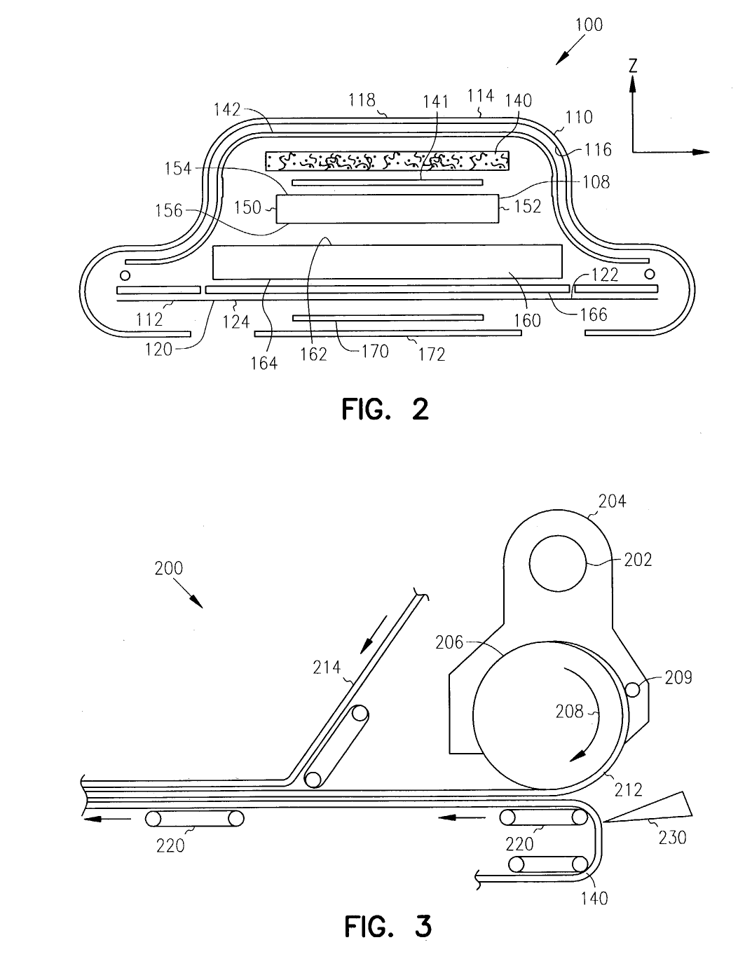

FIG. 1 illustrates one example of an absorbent article 100 according to an embodiment. FIG. 2 illustrates a cross-section taken along A-A of FIG. 1, illustrating an exploded view for clarity. The absorbent article 100 includes a liquid-permeable body side liner 110 and a substantially liquid-impermeable garment side baffle 112, an intake/transfer layer 140, and at least one layer of absorbent material 108. Together, the liquid-permeable body side liner 110 and the substantially liquid-impermeable garment side baffle 112 form an enclosure which contains the intake/transfer layer 140 and the at least one layer of absorbent material 108 therein, as further discussed below. In one embodiment, at least one of the body side liner 110 and the garment side baffle 112 terminates under the absorbent article 100.

The body side liner 110 is presented against the body of the user in a positive-Z direction, assuming the user is standing or sitting, for example. The body side liner 110 includes an outer body side liner surface 114 and an inner body side liner surface 116, where the outer body side liner surface 114 provides a first outer surface 118 of the absorbent article 100.

During use of the absorbent article 100, at least a portion of the garment side baffle 112 is presented against the user's clothing such as the user's underwear. The garment side baffle 112 includes an outer garment side surface 120 and an inner garment side surface 122, where the outer garment side surface 120 provides a second outer surface 124 of the absorbent article 100.

The body side liner 110, in one option, is compliant, soft-feeling, and non-irritating to the wearer's skin. Additionally, the body side liner 110 is liquid pervious to permit liquids to penetrate through its thickness. The body side liner 110 is manufactured from a wide range of materials such as, in one option, woven materials and/or non-woven materials. In another option, the body side liner 110 is derived from, but not limited to, polymeric materials such as apertured formed thermoplastic films, apertured plastic films, hydroformed thermoplastic films, porous foams, reticulated foams, reticulated thermoplastic films, or thermoplastic scrims.

Suitable woven and non-woven materials include natural fibers such as wood or cotton fibers. Other suitable woven and non-woven materials include synthetic fibers such as polymeric fibers including polyester, polypropylene, polyethylene fibers, and the like. Other suitable woven and non-woven material embodiments include a combination of natural and synthetic fibers. It should be noted that one or more of the materials discussed above and below can be used for the body side liner 110.

In one embodiment, the body side liner 110 includes an apertured formed film. An apertured formed film is pervious to body exudates and yet it is non-absorbent and has a reduced tendency to allow liquids to pass back through and re-wet the wearer's skin. Thus, the surface of the formed film which is in contact with the body remains dry, thereby reducing body soiling and creating a more comfortable feel for the wearer.

In one option, the body surface of the body side liner 110 is hydrophilic so as to help liquid to transfer through the body side liner 110 faster than if the body surface was not hydrophilic so as to diminish the likelihood that fluid will flow off the topsheet rather than flowing into and being absorbed by the absorbent core. In one embodiment, surfactant is incorporated into the polymeric materials of the body side liner 110.

Opposite the body side liner 110 is the garment side baffle 112. The garment side baffle 112 is, in one option, substantially impervious to liquids and is manufactured from a thin plastic film, although other flexible liquid impervious materials can also be used. As used herein, the term “flexible” refers to materials which are compliant and will readily conform to the general shape and contours of the human body, and further conforms to movement in the area of use. The garment side baffle 112 assists in preventing exudates absorbed and contained in the absorbent materials within the absorbent article 100 from wetting articles which contact the absorbent article 100 such as pants, pajamas and undergarments.

In one embodiment, the garment side baffle 112 includes at least one of a woven or non-woven material, polymeric films such as thermoplastic films of polyethylene or polypropylene, or composite materials such as a film-coated non-woven material. In another embodiment, the garment side baffle 112 is a polyethylene film having a thickness of from about 0.012 mm (0.5 mil) to about 0.051 mm (2.0 mils). The garment side baffle 112, in one option, permits vapors to escape from the absorbent materials of the absorbent article 100 while still preventing exudates from passing through the garment side baffle 112.

As discussed above, the body side liner 110 and the garment side baffle 112 enclose an intake/transfer layer 140. The intake/transfer layer 140 is disposed between the body side liner 110 and the at least one absorbent material 108, and the intake/transfer layer 140 is in fluid communication with the body side liner 110. A liner adhesive 142 is disposed between the body side liner 110 and the intake/transfer layer 140. The intake/transfer layer 140 directs fluid away from the body side liner 110, by rapidly intaking fluid, and temporarily holding liquid surges, to transport the liquid from its initial entrance point and to substantially completely release the liquid to other parts of the absorbent article 100, for example, by capillary action.

Fibers of the intake/transfer layer 140, in one option, have an orientation in the z-axis. The intake/transfer layer 140 assists in preventing liquid from collecting on a portion of the absorbent article adjacent the wearer's skin. In one option, the intake/transfer layer 140 is not formed of tissue. Furthermore, the intake/transfer layer 140 optionally is formed of hydrophobic material that is treated with a surfactant or processed to impart a predetermined amount of wettability and hydrophilicity. Examples of suitable materials for the intake/transfer layer 140 include, but are not limited to, a meltblown or spun-bonded web of polyolefin fibers, or a bonded-carded-web of natural and synthetic fibers. In another example, the intake/transfer layer 140 is formed of a low-density, lofty, non-woven material which provides a tortuous path for the particulates in the absorbent material. One example of material suitable for the intake/transfer layer 140 is that shown in U.S. Pat. No. 5,336,552, which is incorporated herein by reference. The material is, for example, a bi-component fiber including a polyolefin and ethylene alkyl acrylate copolymer. The intake/transfer layer 140, in another option, includes pores that have meandering or tortuous paths. The tortuous path assists in allowing the intake/transfer layer 140 to be used as a carrier for the absorbent material, as further discussed below, such that the particulate matter will not escape therethrough.

As mentioned above, the body side liner 110 and the garment side baffle 112 enclose at least one absorbent material 108. The absorbent material 108 is a liquid-absorbing medium such as is used in personal care products, such as super absorbent material, or super absorbent material and fluff. The absorbent material 108 can be made from material that tends to swell or expand as it absorbs exudates, including various liquids and/or fluids excreted or exuded by the wearer. In another option, the absorbent material 108 is formed of, or includes, particulate material, such as, but not limited to, odor control materials such as zeolite, graphite, zylitol, or carbon activated materials. The at least one absorbent material 108 includes, in one option, a first absorbent layer 150 that is enclosed by the body side liner 110 and the garment side baffle 112.

The first absorbent layer 150 can be manufactured in a wide variety of sizes and shapes (e.g., rectangular, oval, hourglass, dog bone, asymmetric, etc.) and from a wide variety of liquid-absorbent materials commonly used in absorbent napkins and other absorbent articles. In one option, the first absorbent layer 150 has a first footprint that is the same as or smaller than a footprint of the intake/transfer layer 140. For example, the intake/transfer layer footprint is at least 10% greater than the first footprint. The footprint refers to the surface space occupied by the layer, for example, as defined by the outer perimeter of the layer. In another option, the first absorbent layer 150 has a first width that is the same as or smaller than a width of the intake/transfer layer 140. The footprint for the first absorbent layer 150 can be smaller in length and/or in width.

In one embodiment, the first absorbent layer 150 includes, but is not limited to, one or more of the following: creped cellulose wadding, meltblown polymers, chemically stiffened, modified or cross-linked cellulosic fibers, synthetic fibers such as crimped polyester fibers, peat moss, absorbent foams, absorbent sponges, super absorbent polymers, or absorbent gelling materials. It should be noted that the first absorbent layer 150 includes combinations or subcombinations of these materials, or mixtures of these. In one option, the absorbent layers are formed of particulate material, including materials with relatively small particles, such as odor absorbing material, flakes, or super absorbent material.

In another option, the first absorbent layer 150 includes a combination of fluff and super absorbent material in a variety of weight percentages. For example, the first absorbent layer 150 is, in one option, formed of 70% fluff and 30% super absorbent material. In another option, the first absorbent layer 150 is formed of 75% fluff and 25% super absorbent material. In yet another option, the first absorbent layer 150 is formed of 60-90% fluff, and 40-10% super absorbent material.

In one option, the super absorbent materials includes a product distributed under the name FAVOR SXM 77, FAVOR SXM880, and FAVOR SXM 9543, which are available from Stockhausen GmbH and Co. KC D-47805, Krefeld, Germany. The fluff is formed of, in one example, fluff pulp materials such as crosslinked pulps, hardwood, softwood, and synthetic fibers. An example of suitable fluff is a product distributed under the name CR1654, available from Alliance, and CF 416, available from the Weyerhauser Company.

The first absorbent layer 150, in one option, includes a body side absorbent layer 152. The body side absorbent layer 152 includes a first body side absorbent layer surface 154, and a second body side absorbent layer surface 156. The first body side absorbent layer surface 154 is placed adjacent to the intake/transfer layer 140, for example, with adhesive 141 therebetween.

The article 100 further includes a second absorbent layer 160, for example, a garment side absorbent layer. The second absorbent layer 160 is formed of one or more of the same materials specified for the first absorbent layer 150. Having a second absorbent layer 160 in combination with the first absorbent layer 150 allows for increased control of fluid absorption, as well as increased flexibility in fluff/super absorbent weight percentages, as further discussed below. The second absorbent layer 160 includes a first garment side absorbent layer surface 162 and a second garment side absorbent layer surface 164. In one option, the second absorbent layer 160 is placed in direct, intimate contact with the first absorbent layer 150, with, in another option, no adhesive between the first absorbent layer 150 and the second absorbent layer 160. The first garment side absorbent layer surface 162 is placed, in one option, in direct contact with the second body side absorbent layer surface 156.

Disposed adjacent to the second garment side absorbent layer surface 164 is an adhesive 166. The adhesive 166 is disposed between the second garment side absorbent layer surface 164 and the garment side baffle 112. Furthermore, garment adhesive 170, in one option, is disposed on the outer garment side surface 120, with peel tape 172 disposed thereover. The garment adhesive 170 assists in retaining the absorbent article 100 within a user's garment.

A method for forming the absorbent article includes the use of an apparatus 200, as shown in FIG. 3. The apparatus 200 includes a fiberizer 202 along a processing line, contained in a forming chamber 204 for receiving absorbent material, such as a combination of fluff and super absorbent material. The fiberizer 202 receives and fiberizes the absorbent material. A forming drum 206 rotates generally continuously in the direction of arrow 208. The apparatus 200 optionally includes a scarfing roll 209 which shaves excess absorbent material.

As the forming drum 206 rotates, a first absorbent layer 212 of, for example, fluff/super absorbent material is continuously produced. A conveyor 220, such as a vacuum conveyor, supplies a continuous supply of intake/transfer layer 140 to a location near the forming drum 206. In another option, the intake/transfer layer 140 is provided in segments, or is cut into segments along the processing line. Optionally, an applicator 230 applies a layer of adhesive on the supply of intake/transfer layer 140. The forming drum 206 deposits the first absorbent layer 212 on the intake/transfer layer 140. The intake/transfer layer 140, in one option, includes material with large pores that provide a lofty non-woven tortuous path that prevents the shakeout of the super absorbent material as the materials travel on the conveyor 220 and, as the intake/transfer layer 140 and the first absorbent layer 212 continue down along the processing line for further processing, for example, for debulking and cutting, or embossing. One example of suitable material for the intake/transfer layer 140 is that described in U.S. Pat. No. 5,336,552, which is incorporated herein by reference.

It should be noted that the intake/transfer layer 140 is not wrapped around the fluff/super absorbent material. Furthermore, no other layers are placed on the opposite side of the first absorbent layer 212 to encompass the first absorbent layer 212. A second absorbent layer 214 is placed on the first absorbent layer 212 such that the two absorbent layers 212, 214 are placed in intimate contact with one another. That is, a surface of the first absorbent layer 212 directly contacts a surface of the second absorbent layer 214. The intake/transfer layer 140 acts as a carrier for the first absorbent layer 212, and prevents the particulate material of the first absorbent layer from escaping the first intake layer surface.

The method allows for the removal of the tissue layer or a separate envelope of material from being placed around the super absorbent material. This provides for increased ways to cut the absorbent article during further processing, since fewer layers will be present, while maintaining the high absorption features. Furthermore, the direct contact between the layers within the absorbent article, for example, without the presence of tissue, provides for enhanced fluid acquisition within the absorbent article, and/or improved bonding between the layers, for example, by adhesive or embossing.

In the foregoing Detailed Description, various features are grouped together in a single embodiment for the purpose of streamlining the disclosure. This method of disclosure is not to be interpreted as reflecting an intention that the claimed embodiments of the invention require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separate embodiment.

It is to be understood that the above description is intended to be illustrative, and not restrictive. Many other embodiments will be apparent to those of skill in the art upon reading and understanding the above description. It should be noted that embodiments discussed in different portions of the description or referred to in different drawings can be combined to form additional embodiments of the present application. The scope of the invention should, therefore, be determined with reference to the appended claims, along with the full scope of equivalents to which such claims are entitled.

What is claimed is:

1. An absorbent article comprising:
a moisture permeable body side liner having an outer body side liner surface and an inner body side liner surface, the outer body side liner surface providing a first outer surface of the absorbent article;
an intake/transfer layer having a first intake layer surface and a second intake layer surface, the second intake layer surface opposite the first intake layer surface, the intake/transfer layer disposed in fluid communication with the moisture permeable body side liner, the intake/transfer layer having a first footprint;
a body side absorbent layer including particulate material in fluid communication with the intake/transfer layer, the body side absorbent layer having a first side contacting the second intake surface, the body side absorbent layer having a second footprint, the first footprint is at least as large as the second footprint;
a garment side absorbent layer in fluid communication with the body side absorbent layer; and
a substantially moisture impermeable garment side baffle having an outer garment side surface and an inner garment side surface, the outer garment side surface providing a second outer surface of the absorbent article.

2. The absorbent article as recited in claim 1, wherein the intake/transfer layer is formed of a bi-component fiber.

3. The absorbent article as recited in claim 2, wherein the bi-component fiber includes a polyolefin and ethylene alkyl acrylate copolymer.

4. The absorbent article as recited in claim 1, wherein the intake/transfer layer is formed of lofty, non-woven material.

5. The absorbent article as recited in claim 1, wherein the particulate material includes super absorbent material.

6. The absorbent article as recited in claim 5, wherein at least one of the body side absorbent layer and the garment side absorbent layer includes about 70% fluff material and about 30% super absorbent material by weight.

7. The absorbent article as recited in claim 1, wherein the first footprint is larger than the second footprint.

8. The absorbent article as recited in claim 7, wherein the first footprint is at least 10% larger than the second footprint.

9. An absorbent article comprising:
a moisture permeable body side liner having an outer body side liner surface and an inner body side liner surface, the outer body side liner surface providing a first outer surface of the absorbent article;
an intake/transfer layer having a first intake layer surface and a second intake layer surface, the second intake layer surface opposite the first intake layer surface, the first intake layer surface directed toward the body side liner, the intake/transfer layer disposed in fluid communication with the moisture permeable body side liner, the intake/transfer layer having a first footprint;
dual absorbent layers including particulate material including a first absorbent layer and a second absorbent layer, a first surface of the first absorbent layer in contact with the intake/transfer layer where the first absorbent layer is carried by the intake/transfer layer, the intake/transfer layer preventing the particulate material from escaping the first intake layer surface, the first absorbent layer having a second footprint, where the first footprint is at least as large as the second footprint; and
a substantially moisture impermeable garment side baffle having an outer garment side surface and an inner garment side surface, the outer garment side surface providing a second outer surface of the absorbent article, the inner garment side surface in contact with the second surface of the second absorbent layer.

10. The absorbent article as recited in claim 9, wherein the intake/transfer layer the first footprint is larger than the second footprint.

11. The absorbent article as recited in claim 9, further comprising a layer of adhesive disposed between the intake/transfer layer and the first surface of the first absorbent layer.

12. The absorbent article as recited in claim 9, wherein the intake/transfer layer is not formed of tissue.

13. A method for forming an absorbent article, the method comprising:
forming a particulate material on a processing line at a forming location;
providing a layer of intake/transfer material proximate to the forming location;
conveying the layer of intake/transfer material along a conveyor;
forming a first layer of absorbent material from the particulate material; and
depositing the first layer of absorbent material on the layer of intake/transfer material on the conveyor.

14. The method as recited in claim 13, wherein forming the particulate material includes mixing super absorbent material with fluff.

15. The method as recited in claim 13, further comprising depositing a layer of adhesive on the layer of intake/transfer material prior to forming the first layer of absorbent material on the layer of intake/transfer material.

16. The method as recited in claim 13, further comprising depositing a second layer of absorbent material on the first layer of absorbent material.

17. The method as recited in claim 16, wherein depositing the second layer of absorbent material includes depositing the second layer of absorbent material directly on the first layer of absorbent material.

18. The method as recited in claim 13, wherein conveying the layer of intake/transfer material includes conveying the layer of intake/transfer material along a vacuum conveyor.

19. The method as recited in claim 13, wherein depositing the first layer of absorbent material on the layer of intake/transfer material includes depositing the first layer of absorbent material having a footprint the same as or smaller than an intake/transfer layer footprint.

20. The method as recited in claim 12, wherein providing the layer of intake/transfer material includes providing a continuous layer of intake/transfer material.
